(19) United States
(12) Patent Application Publication
(10) Pub. No.: US 2017/0079574 A1
(43) Pub. Date: Mar. 23, 2017

(54) Wearable technology that monitors health and safety

(71) Applicants: Daniela Stefanny Rodriguez Restrepo, Harrisburg, PA (US); Justin Ryan Penney, North Andover, MA (US); Jennifer Patricia Penunuri, Newport Beach, CA (US); Haley Elizabeth Yeranossian, Ridgefield, CT (US); Matthew Richard Ehde, Minoa, NY (US)

(72) Inventors: Daniela Stefanny Rodriguez Restrepo, Harrisburg, PA (US); Justin Ryan Penney, North Andover, MA (US); Jennifer Patricia Penunuri, Newport Beach, CA (US); Haley Elizabeth Yeranossian, Ridgefield, CT (US); Matthew Richard Ehde, Minoa, NY (US)

(21) Appl. No.: 14/862,105

(22) Filed: Sep. 22, 2015

(51) Int. Cl. A61B 5/00; A61B 5/11

(52) U.S. Cl. CPC A61B 5/4266; A61B 5/4845; A61B 5/002; A61B 5/6802; A61B 5/11; A61B 2562/0219; A61B 2503/10

(57) ABSTRACT

Wearable technology for individuals containing a transdermal sweat sensor, Bluetooth low energy micro-chip, and accelerometer connected to a smartphone application. The transdermal sweat sensor detects perspiration on the skin and determines the blood alcohol content (BAC) as well as ingestion of a date-rape drug by the individual. This reading is transmitted to the smartphone application via the Bluetooth low energy micro-chip, which allows the individual and three emergency contacts to monitor the BAC and drug ingestion, on the smartphone application, to determine if the individual wearing the bracelet is in distress. The Bluetooth low energy micro-chip will also provide the geographic location of the individual to the emergency contacts in order to provide aid. Additionally, the accelerometer will also serve as a step and calorie counter. The goal of the bracelet is to reduce the risk of sexual assaults associated with over consumption of alcohol and date-rape drugs.

### CROSS-REFERENCE TO RELATED APPLICATIONS

U.S. Pat. No. 5,944,661
Potential and diffusion controlled solid electrolyte sensor for continuous measurement of very low levels of transdermal alcohol

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF INVENTION

This invention relates to a device that is used in daily life for health, fitness, and safety. This new wearable technology monitors calories, steps taken, blood alcohol content (BAC), and traces of date rape drugs. The wearer can connect this invention to an app in their smartphone that allows them to select emergency contacts of their own choosing who would then receive alerts and location information of the wearer in instances where the individual could find themselves in potentially dangerous situations. FIGS. 1-5 provide a visual representation of the wearable technology itself. The use for this wearable technology is to reduce the risk of date rape and sexual assault, while promoting a healthy lifestyle.

### BRIEF SUMMARY OF THE INVENTION

The invention falls under wearable technology that monitors health and fitness through a biometric monitoring device which consists of an accelerometer, vibration motor hard drive, battery, Bluetooth low energy chip, and transdermal sweat sensor that relays data back to a smartphone application. The advantage from this technology is to promote a healthy lifestyle and safety by alerting individuals of calories burned, steps taken, high blood alcohol levels and/or date rape drugs through the individuals perspiration. The purpose is to reduce the risk of being taken advantage of when individuals are exposed to alcohol and/or date rape drugs.

### BRIEF DESCRIPTION OF THE SEVERAL VIEWS OF THE DRAWINGS

FIG. 1 illustrates a perspective of the wearable technology according to an embodiment of the invention

FIG. 2 illustrates an aerial view of the wearable technology according to an embodiment of the invention

FIG. 3 illustrates the biometric monitoring device according to an embodiment of the invention and the contents of the biometric monitoring device according to an embodiment of the invention

FIG. 4 illustrates how to use the wearable technology according to an embodiment of the invention

FIG. 5 illustrates the smartphone application according to an embodiment of the invention

### DETAILED DESCRIPTION OF THE INVENTION

The following detailed description represents the best currently contemplated modes for carrying out the invention. The description is not to be taken in a limiting sense, but is made merely for the purpose of illustrating the general principles of the invention.

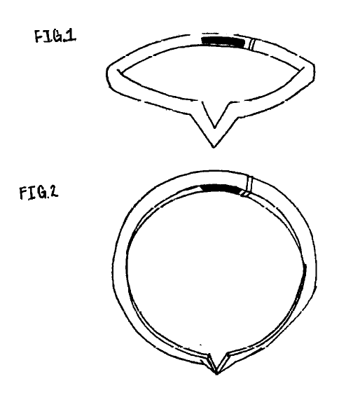

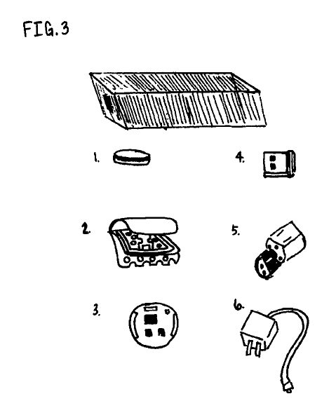

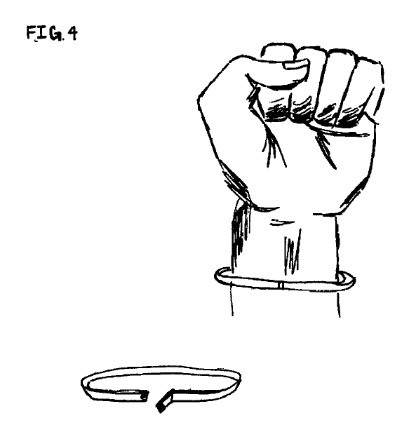

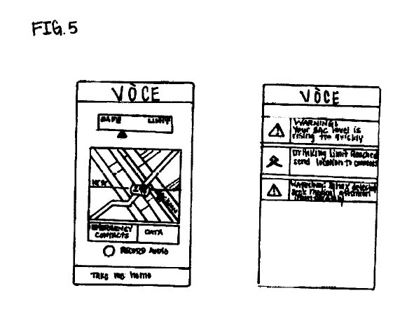

The invention relates to health and safety. It is suitable for anyone who is interested in a healthy lifestyle and protection against attackers involving alcohol and/or date rape drugs. This wearable technology incorporates multiple types of equipment. Referring to FIG. 3, a biometric monitoring device approximately 1 cm wide and 1.5 inches long will include a 3.1 battery 3.2 transdermal sweat sensor motion detection, 3.3 Bluetooth low energy chip 3.4 vibration motor hard drive, and 3.5 accelerometer into one small light box that fits into a wearable case shown in FIGS. 1 and 2. This is so the person wearing the device finds the design comfortable. The design of the wearable technology varies from shapes and sizes to adapt for the user (e.g. bracelets, necklaces, rings, etc.). The material for this design is approximately 5.5-8.2 inches long and 0.8 cm wide, shown in FIGS. 1 and 2 made of a metal that is hypoallergenic, (e.g. nickel free stainless steel, nickel free silver, gold, etc.) To use this invention one must wear the technology so that it is touching their skin shown in FIG. 4. There is a clasp that when closed by the user, activates the FIG. 3 monitoring device, best viewed in FIG. 4. When turned on the wearable technology vibrates via the 3.4 vibration motor to signal it is activated. The wearable technology interconnects with a 3.6 charger through a connector. That person must also connect the smart phone application shown in FIG. 5 and personalize their settings.

The most notable integrated technologies are a 3.2 transdermal sweat sensor and a 3.3 Bluetooth low energy microchip. In addition, the wearable technology also has an 3.5 accelerometer that acts as a step and calorie counter. The 3.2 transdermal sweat sensor detects alcohol levels and date rape drugs, (e.g. Xanax, rohypnol, etc.). The 3.3 Bluetooth low energy chip uses geo-tagging technology to precisely locate the user as well as connects with the 3.2 transdermal sensor by a smartphone application shown in FIG. 5. When the 3.2 transdermal sensor is “in distress” (when high levels of alcohol or drugs are detected) it will connect with the 3.3 Bluetooth chip to send push-notifications to the users smartphone application in FIG. 5. The data is displayed as a range from green to red in FIG. 5. The data ranges in the following order: Green=Safe (level 0.0 to 0.019), Yellow=Caution (level 0.02 to 0.039), Orange=Hazardous (level 0.04 to 0.079) and lastly, Red=Dangerous (level 0.08 and above) shown in FIG. 5.

While the invention has been particularly shown and described with respect to the illustrated embodiments thereof, it will be understood by those skilled in the art that the foregoing and other changes in form and details may be made therein without departing from the spirit and scope of the invention

1. A wearable technology device for promoting health, fitness. and safety comprising:
an apparatus having a battery, a transdermal sweat sensor, motion detection, Bluetooth low energy chip, vibration motor, hard drive, and an accelerometer;
said apparatus is put together in a biometric light weight cube of approximately 1 cm wide and 1.5 inches long;
the Bluetooth low energy chip in said apparatus connects to a smartphone application in order to act as the means of connecting the wearer to the users selected contact.

2. The wearable technology device as in claim 1 wherein said transdermal sweat sensor and said Bluetooth low energy chin in said apparatus connect to detect high levels of alcohol and/or any date rape drugs in the users skin by perspiration:
said transdermal sweat sensor detects alcohol and/or date rape drugs found in users perspiration triggers the Bluetooth low energy chin in said apparatus to give off a signal that uses GPS to precisely locate the user coordinates;
said Bluetooth low energy chip signal will be identified by the associated said application as in on the user's smartphone through the phone's operating system.

3. The wearable technology device as in claim 1 wherein said accelerometer in said apparatus and said Bluetooth low energy chip in said apparatus connect to monitor health and fitness by monitoring steps taken and counting calories:
said Bluetooth low energy chip signal will be identified by the associated said application as in on the user's smartphone through the phone's operating system.
